(19) United States
(12) Patent Application Publication
(10) Pub. No.: US 2021/0330232 A1
(43) Pub. Date: Oct. 28, 2021

(54) ELECTRODE AND USES THEREOF

(71) Applicant: BioSerenity, Paris (FR)

(72) Inventors: Julie Oziat, Paris (FR); Philippe GUERMONPREZ, Hazebrouck (FR)

(73) Assignee: BioSerenity, Paris (FR)

(21) Appl. No.: 17/286,843

(22) PCT Filed: Oct. 24, 2019
(86) PCT No.: PCT/EP2019/079022

§ 371 Date: Apr. 20, 2021

(30) Foreign Application Priority Data
Oct. 24, 2018 (EP) 18306386.6

(51) Int. Cl. A61B 5/257

(52) U.S. Cl. CPC A61B 5/257; A61B 2560/0285; A61B 2562/0215

(57) ABSTRACT

The present invention relates to an electrode (1) comprising: —at least one reservoir (11) comprising a peripheral wall (111) made of a foam material, wherein said reservoir (11) is filled with a viscous electrolytic paste (12); —at least one plate (13) comprising a conductor material, wherein said plate (13) is in contact with the electrolytic paste (12) and configured to conduct electrical signals to a recording device; —at least one fastener (14); and wherein the at least one reservoir (11) comprises a hole (113) configured to release the electrolytic paste (12) under pressure applied on the electrode (1). The present invention also relates to the use of said electrode (1) and a process for the implementation of an electrode (1).

### FIELD OF INVENTION

The present invention relates to the field of electrodes for measuring electrophysiological signals. In particular, the present invention relates to the field of EEG, ECG, FECG, EMG, EIT, TENS or bioimpedance electrodes.

### BACKGROUND OF INVENTION

To measure electrophysiological signals on a subject, it can be requested to have an array of electrodes distributed on the body surface. In this case, it is difficult to set up such a network. If the electrodes are sticky electrodes (like standard ECG electrodes) and the support is stretchable, it is very difficult to place all the electrodes at the same time at the exact morphological position on the body. If the electrodes are gel electrodes, the same problem occurs: the gel will settle all over the skin and induce short circuits.

In particular, in the case of certain pathologies of the brain, it is necessary to monitor the electrical activity of the brain (EEG) for a long time and especially during sleep.

During EEG monitoring, the electrodes are affixed to the scalp of the subject. However, currently used electrodes are either uncomfortable electrodes that do not allow sleep or require lengthy and technical manipulations.

Indeed, currently used electrodes comprise rigid materials intended to be in contact with the scalp, those materials allowing no comfort to the subject. They also need to be filled with an electrolytic paste after placement on the scalp, bringing time consuming and burdensome manipulations of the electrode during use.

The object of this invention is to provide an electrode that can be placed in the exact position on the body of a subject and then release an electrolytic paste on demand by pressure. Said electrode is ready to use in an EEG electrode support, an ECG electrode support, a FECG electrode support, an EMG electrode support, an EIT electrode support, a TENS electrode support, a bioimpedance electrode support, or any electrostimulation electrode support, comfortable and efficient for measuring the electrophysiological signals of a subject over long periods and particularly during sleep.

### SUMMARY

The present invention relates to an electrode comprising:
at least one reservoir comprising a peripheral wall made of a foam material, wherein said reservoir is filled with an electrolytic paste, wherein the electrolytic paste (12) exhibits a viscosity ranging from 10 Pa s to 30 kPa s;
at least one plate comprising a conductor material, wherein said plate is in contact with the electrolytic paste and configured to conduct electrical signals to a recording device;
at least one fastener; and

wherein the at least one reservoir comprises a hole configured to release the electrolytic paste under pressure applied on the electrode.

The electrode is pre-filled with the electrolytic paste, thus ready to use in an EEG electrode support such as for example an EEG headgear, an ECG electrode support, a FECG electrode support, an EMG electrode support, an EIT electrode support, a TENS electrode support, a bioimpedance electrode support, or any electrostimulation electrode support for example. It does not necessitate a filling after it has been placed on the body, therefore one hole for the delivery of the electrolytic paste to the skin (the scalp for example) is sufficient instead of two holes (one of them for the filling) in conventional electrodes.

The plate allows electrical communication with a subject's body by conducting the electrophysiological signals from the skin to a recording device.

The plate allows electrical communication with a subject's scalp by conducting the EEG signals from the scalp to a recording device.

In one embodiment, the electrode is configured to measure an electrophysiological signal, an electroencephalograph (EEG) signal, an electrocardiograph (ECG) signal, or a bioimpedance.

In one embodiment, the electrode is an EEG electrode, an ECG electrode, a FECG electrode, an EMG electrode, an EIT electrode, a TENS electrode, an electrode for bioimpedance measurement or an electrode for electrostimulation.

In one embodiment, the electrode further comprises a bottom polymeric washer.

In one embodiment, the electrode further comprises at least one double-sided adhesive sheet between the peripheral wall and the bottom polymeric washer.

In one embodiment, the at least one double-sided adhesive sheet comprises a material such as for example 3M double-sided bonding tape, a layer of glue sandwiched between two non-adhesive protective sheets that are removed when used, or any other double-sided adhesive sheet known in the art.

In one embodiment, the electrode is configured to measure the cerebral activity of a subject, i.e. the electrode is configured to measure EEG signals of a subject.

In one embodiment, the electrode is configured to measure the cardiac activity of a subject, i.e. the electrode is configured to measure ECG signals of a subject.

In one embodiment, the electrode is configured to measure the fetal cardiac activity of a subject, i.e. the electrode is configured to measure FECG signals of a subject.

In one embodiment, the electrode is configured to measure EMG signals of a subject.

In one embodiment, the electrode is configured to measure the bioimpedance of a subject.

In one embodiment, the electrode is configured to measure the electrical conductivity, permittivity, and impedance of a subject.

In one embodiment, the electrode is a passive or an active electrode.

In one embodiment, the electrode is a semi-dry conductive electrode comprising an ionic gel; said ionic gel being the electrolytic paste.

In one embodiment, the electrode is disposable. In this embodiment, the disposability ensures a good hygiene and saves time. In this embodiment, the electrode is changed after every use.

According to one embodiment, the electrode is disinfectable. In this embodiment, the electrode can be disinfected using a cleaning bath, a disinfectant wipe, or any other means known by one skilled in the art.

In one embodiment, the electrode is adapted to be received in and to cooperate with an EEG electrode support such as for example an EEG headgear, an ECG electrode support, a FECG electrode support, an EMG electrode support, an EIT electrode support, a TENS electrode support, a bioimpedance electrode support, or any electrostimulation electrode support.

In one embodiment, the electrode is ready to use on an EEG electrode support such as for example an EEG electrode headgear, an ECG electrode support, a FECG electrode support, an EMG electrode support, an EIT electrode support, a TENS electrode support, a bioimpedance electrode support, or any electrostimulation electrode support.

In one embodiment, the EEG headgear is a headwear, a headset, a hat, a helmet, a cap, or any other EEG headgear known in the art.

In one embodiment, the at least one reservoir has a volume ranging from 0.1 ml to 100 ml, from 1 ml to 100 ml, from 5 ml to 100 ml, from 10 ml to 100 ml, from 20 ml to 100 ml, from 30 ml to 100 ml, from 40 ml to 100 ml, from 50 ml to 100 ml, from 60 ml to 100 ml, from 70 ml to 100 ml, from 80 ml to 100 ml, or from 90 ml to 100 ml. In one embodiment, the at least one reservoir has a volume ranging from 0.1 ml to 90 ml, from 0.1 ml to 80 ml, from 0.1 ml to 70 ml, from 0.1 ml to 60 ml, from 0.1 ml to 50 ml, from 0.1 ml to 40 ml, from 0.1 ml to 30 ml, from 0.1 ml to 20 ml, from 0.1 ml to 10 ml, or from 0.1 ml to 5 ml.

The reservoir is pre-filled with the electrolytic paste, i.e. the reservoir is filled before use of the electrode, avoiding time consuming and burdensome manipulations or preparations of the electrode during use.

The reservoir is configured to store enough electrolytic paste to maintain electrical continuity between the skin and the plate for at least 12 hours, at least 14 hours, at least 16 hours, at least 18 hours, at least 20 hours, at least 22 hours or at least 24 hours.

In one embodiment, the at least one reservoir has a height ranging from 1 mm to 50 mm, from 1 mm to 40 mm, from 1 mm to 30 mm, from 1 mm to 20 mm, from 1 mm to 10 mm, from 5 mm to 50 mm, from 10 mm to 50 mm, from 20 mm to 50 mm, from 30 mm to 50 mm, or from 40 mm to 50 mm.

In one embodiment, the at least one reservoir has a diameter ranging from 3 mm to 70 mm, from 3 mm to 60 mm, from 3 mm to 50 mm, from 3 mm to 40 mm, from 3 mm to 30 mm, from 3 mm to 20 mm, from 3 mm to 10 mm, from 5 mm to 70 mm, from 10 mm to 70 mm, from 20 mm to 70 mm, from 30 mm to 70 mm, from 40 mm to 70 mm, from 50 mm to 70 mm, or from 60 mm to 70 mm.

In one embodiment, the hole has a diameter ranging from 0.5 mm to 30 mm, from 0.5 mm to 20 mm, from 0.5 mm to 15 mm, from 0.5 mm to 10 mm, from 0.5 mm to 5 mm, from 1 mm to 30 mm, from 5 mm to 30 mm, from 10 mm to 30 mm, from 15 mm to 30 mm, from 20 mm to 30 mm, or from 25 mm to 30 mm.

In one embodiment, when the electrode is placed in an electrode support, the hole is oriented toward the skin of the subject wearing said electrode support.

Herein, the electrode support refers to an electrode array support, an EEG electrode support such as for example an EEG headgear, an ECG electrode support, a FECG electrode support, an EMG electrode support, an EIT electrode support, a TENS electrode support, a bioimpedance electrode support, or any electrostimulation electrode support.

In one embodiment, when the electrode is placed in an EEG headgear, the hole is oriented toward the scalp of the subject wearing said EEG headgear.

In one embodiment, the peripheral wall is a shell, a frame, a case, a cone, or a cylinder or any other convenient shape.

In one embodiment, the peripheral wall has a height ranging from 1 mm to 50 mm, from 1 mm to 40 mm, from 1 mm to 30 mm, from 1 mm to 20 mm, from 1 mm to 10 mm, from 5 mm to 50 mm, from 10 mm to 50 mm, from 20 mm to 50 mm, from 30 mm to 50 mm, or from 40 mm to 50 mm.

In one embodiment, the peripheral wall has an inner diameter ranging from 2 mm to 50 mm, from 2 mm to 40 mm, from 2 mm to 30 mm, from 2 mm to 20 mm, from 2 mm to 10 mm, from 5 mm to 50 mm, from 10 mm to 50 mm, from 20 mm to 50 mm, from 30 mm to 50 mm, or from 40 mm to 50 mm.

In one embodiment, the peripheral wall has an outer diameter ranging from 3 mm to 70 mm, from 3 mm to 60 mm, from 3 mm to 50 mm, from 3 mm to 40 mm, from 3 mm to 30 mm, from 3 mm to 20 mm, from 3 mm to 10 mm, from 5 mm to 70 mm, from 10 mm to 70 mm, from 20 mm to 70 mm, from 30 mm to 70 mm, from 40 mm to 70 mm, from 50 mm to 70 mm, or from 60 mm to 70 mm.

In one embodiment, the peripheral wall has a volume ranging from 0.1 ml to 100 ml, from 1 ml to 100 ml, from 5 ml to 100 ml, from 10 ml to 100 ml, from 20 ml to 100 ml, from 30 ml to 100 ml, from 40 ml to 100 ml, from 50 ml to 100 ml, from 60 ml to 100 ml, from 70 ml to 100 ml, from 80 ml to 100 ml, or from 90 ml to 100 ml. In one embodiment, the peripheral wall 111 has a volume ranging from 1 ml to 90 ml, from 1 ml to 80 ml, from 1 ml to 70 ml, from 1 ml to 60 ml, from 1 ml to 50 ml, from 1 ml to 40 ml, from 1 ml to 30 ml, from 1 ml to 20 ml, from 1 ml to 10 ml, or from 1 ml to 5 ml.

In one embodiment, the peripheral wall comprises at least two parts assembled with a double-sided adhesive sheet.

In one embodiment, the peripheral wall comprises two parts assembled with a double-sided adhesive sheet.

In one embodiment, the foam material is pliable, deformable, flexible, and/or resilient. In this embodiment, the foam material can be deformed without breaking upon pressure on the fastener of the electrode so that the electrolytic paste can be delivered without damaging the reservoir. The pressure exerted on the fastener is a pressure tolerable for the human body.

The electrolytic paste has a sufficiently high viscosity not to flow out of the reservoir without an external mechanical action and not to be absorbed by the foam material, so as to flow out of the reservoir with an external mechanical action.

In one embodiment, the foam material is a shape-memory material. This embodiment is particularly advantageous as the foam material remembers its original shape and will return to it after deformation.

In one embodiment, the foam material is an anti-static foam. This embodiment is advantageous as static electricity could be an issue during measurement of an electrophysiological signal.

In one embodiment, the foam material is a low, medium density or a high-density material.

In one embodiment, the foam material has a density ranging from 15 to 250 kg/m³, from 20 to 250 kg/m³, from 25 to 250 kg/m³, from 30 to 250 kg/m³, from 40 to 250 kg/m³, from 45 to 250 kg/m³, from 50 to 250 kg/m³, from 60 to 250 kg/m³, from 70 to 250 kg/m³, from 80 to 250 kg/m³, from 90 to 250 kg/m³, from 100 to 250 kg/m³, from 120 to 250 kg/m³, from 140 to 250 kg/m³, from 160 to 250 kg/m³, from 180 to 250 kg/m³, from 200 to 250 kg/m³, from 220 to 250 kg/m³, from 15 to 220 kg/m³, from 15 to 200 kg/m³, from 15 to 180 kg/m³, from 15 to 160 kg/m³, from 15 to 140 kg/m³, from 15 to 120 kg/m³, from 15 to 100 kg/m³, from 15 to 90 kg/m³, from 15 to 80 kg/m³, from 15 to 70 kg/m³, from 15 to 60 kg/m³, from 15 to 50 kg/m³, from 15 to 40 kg/m³, or from 15 to 30 kg/m³.

In one embodiment, the foam material is an open cell foam.

In one embodiment, the foam material is a closed cell foam. This is advantageous as a closed cell foam does not absorb water and can therefore be stored without concerns of environmental humidity.

In one embodiment, the foam material is biocompatible, antimicrobial and/or non-allergenic. A biocompatible material is advantageous as it allows contact with the skin of a subject. An antimicrobial and/or non-allergenic material is advantageous as it prevents growth of undesirable microorganisms and/or allergy upon contact with skin. As the electrode is destined to be used in contact with the skin of a subject (notably on the head), it is important that the foam material exhibits such properties.

In one embodiment, the foam material is a polymer, such as for example an organic polymer or an inorganic polymer.

In one embodiment, the foam material comprises a material selected in the group of: polyurethane, silicone, polyethylene, or a mixture thereof.

In one embodiment, the foam material has at least one sticky face configured to adhere to the skin. This embodiment is particularly advantageous as the foam material can adhere to the skin and/or scalp of a subject, thus avoiding inopportune movement of the electrode on the skin and avoiding leaks of electrolytic paste.

In one embodiment, the bottom polymeric washer is made of a non-conductive plastic. In this embodiment, the non-conductive plastic is chosen because it is nonconductive, flexible, cheap, bio-compatible, easy to process. In one embodiment, the non-conductive plastic is preferably a material with a high Young modulus.

In one embodiment, the bottom polymeric washer comprises a material selected in the group of: acetate, polylactic acid (PLA), acrylonitrile butadiene styrene (ABS), polyethylene (PE), polyamide, or a mixture thereof.

In one embodiment, the bottom polymeric washer has a flattened shape.

In one embodiment, the electrolytic paste exhibits an electrical conductivity ranging from 0.01 to 100 S/m, from 0.1 to 100 S/m, from 0.5 to 100 S/m, from 1 to 100 S/m, from 5 to 100 S/m, from 10 to 100 S/m, from 20 to 100 S/m, from 30 to 100 S/m, from 40 to 100 S/m, from 50 to 100 S/m, from 60 to 100 S/m, from 70 to 100 S/m, from 80 to 100 S/m, from 90 to 100 S/m, from 0.01 to 90 S/m, from 0.01 to 80 S/m, from 0.01 to 70 S/m, from 0.01 to 60 S/m, from 0.01 to 50 S/m, from 0.01 to 40 S/m, from 0.01 to 30 S/m, from 0.01 to 20 S/m, from 0.01 to 10 S/m, from 0.01 to 5 S/m, from 0.01 to 1 S/m, or from 0.01 to 0.5 S/m.

The electrolytic paste is sufficiently conductive and the diameter of the reservoir is sufficiently large so that the impedance across the electrode remains low enough to maintain a good signal.

In the invention, the electrolytic paste exhibits a viscosity ranging from 10 Pa·s to 30 kPa·s. In some embodiments, the electrolytic paste exhibits a viscosity ranging from 10 kPa·s to 30 kPa·s, from 10 kPa·s to 25 kPa·s, from 10 kPa·s to 20 kPa·s, from 10 kPa·s to 15 kPa·s, from 15 kPa·s to 30 kPa·s, from 20 kPa·s to 30 kPa·s, or from 25 kPa·s to 30 kPa·s. In other embodiments, the electrolytic paste exhibits a viscosity ranging from 10 Pa·s to 10 kPa·s, from 10 Pa·s to 1 kPa·s, from 10 Pa·s to 250 Pa·s, from 10 Pa·s to 150 Pa·s, from 100 Pa·s to 10 kPa·s, from 100 Pa·s to 1 kPa·s, or from 1 kPa·s to 10 kPa·s.

The electrolytic paste has a sufficiently high viscosity not to flow out of the reservoir without an external mechanical action. In addition, the high viscosity of the electrolytic paste prevents said electrolytic paste to be absorbed by a foam material. When electrolytic paste flows out of the reservoir with an external mechanical action, electrolytic paste does not flow into the peripheral wall of the reservoir. Hence, the peripheral wall made of a foam material is a barrier for the electrolyte gel. In other words, an electrolytic paste with a viscosity ranging from 10 Pa·s to 30 kPa·s cannot be stored in (nor released from) a reservoir consisting of an open cell foam material.

Besides, the viscosity of the electrolytic paste is chosen so that it can exit the reservoir under manual pressure, enter in contact with the skin through the hair while not flowing over the long term which would cause short circuits between electrodes while maintaining electrical continuity inside the electrode.

In one embodiment, the electrolytic paste is compatible with skin contact.

In one embodiment, the electrolytic paste is biocompatible.

In one embodiment, the electrolytic paste is an ionic gel.

In one embodiment, the electrolytic paste has an electrically conductive gel composition.

In one embodiment, examples of the electrolytic paste include but are not limited to: electrolyte cream or gel, electrode cream or gel, ECG cream or gel, electrode cream or gel for EEG, ECG, FECG, EMG, EIT, TENS or bioimpedance measurement.

In one embodiment, the electrolytic paste is a hydrogel, preferably an electrically conductive hydrogel.

In one embodiment, the conductor material of the at least one plate is an electrode material.

In one embodiment, the conductor material of the at least one plate is a redox couple such as for example a metal/metallic salt couple.

In one embodiment, the conductor material of the at least one plate is Ag/AgCl, electrically conductive silicone, electrically conductive polymer, a plastic loaded with a conductive material such as for example a plastic coated with Ag/AgCl, or mixture thereof.

In one embodiment, the plate has a flattened shape.

In one embodiment, the plate is a flattened cylinder, a disk, a pellet, a tablet, a flattened square, or any other convenient design.

In one embodiment, the plate has a height ranging from 0.1 to 40 mm, from 1 to 40 mm, from 5 to 40 mm, from 10 to 40 mm, from 15 to 40 mm, from 20 to 40 mm, from 25 to 40 mm, from 30 to 40 mm, from 35 to 40 mm, from 0.1 to 35 mm, from 0.1 to 30 mm, from 0.1 to 25 mm, from 0.1 to 20 mm, from 0.1 to 10 mm, from 0.1 to 5 mm, from 0.1 to 1 mm, or from 0.1 to 0.5 mm.

In one embodiment, the plate has a diameter ranging from 1 mm to 50 mm, from 1 mm to 40 mm, from 1 mm to 30 mm, from 1 mm to 20 mm, from 1 mm to 10 mm, from 5 mm to 50 mm, from 10 mm to 50 mm, from 20 mm to 50 mm, from 30 mm to 50 mm, or from 40 mm to 50 mm.

In one embodiment, the at least one fastener is configured to attach the electrode to an EEG electrode support such as for example an EEG headgear, an ECG electrode support, a FECG electrode support, an EMG electrode support, an EIT electrode support, a TENS electrode support, a bioimpedance electrode support, or any electrostimulation electrode support.

In one embodiment, the at least one fastener is a snap fastener, a hook and loop fastener, a button, a clip, or a locking groove.

In one embodiment, the at least one fastener is a female or a male snap fastener.

In one embodiment, the components of the electrode are glued, or fixed together.

The present invention also relates to the use of the electrode of the invention to measure an electrophysiological signal, an EEG signal, an ECG signal, an EMG signal, an FECG signal, an EIT signal or a bioimpedance.

The present invention also relates to the use of the electrode of the invention in an ECG electrode support. The electrode is therefore used to measure ECG signals of the subject wearing the ECG electrode support.

The present invention also relates to the use of the electrode of the invention in an EMG electrode support. The electrode is therefore used to measure EMG signals of the subject wearing the EMG electrode support.

The present invention also relates to the use of the electrode of the invention in an FECG electrode support. The electrode is therefore used to measure FECG signals of the subject wearing the FECG electrode support.

The present invention also relates to the use of the electrode of the invention in an EIT electrode support. The electrode is therefore used to measure EIT signals of the subject wearing the EIT electrode support.

The present invention also relates to the use of the electrode of the invention in a bioimpedance electrode support. The electrode is therefore used to measure bioimpedance of the subject wearing the bioimpedance electrode support.

In one embodiment, the electrode is used in an EEG electrode support, an ECG electrode support, an EMG electrode support, a FECG electrode support, an EIT electrode support, bioimpedance electrode support or any electrostimulation electrode support.

In one embodiment, the electrode is clipped, fastened, connected, attached, or fixed on or in the EEG electrode support, an ECG electrode support, an EMG electrode support, a FECG electrode support, an EIT electrode support, bioimpedance electrode support or electrostimulation support.

The present invention also relates to the use of the electrode of the invention in an EEG headgear. The electrode is therefore used to measure EEG signals of the subject wearing the EEG headgear.

The EEG headgear is as described hereabove.

In one embodiment, the electrode is clipped, fastened, connected, attached, or fixed on or in the EEG headgear.

According to one embodiment, the electrode is removable. In this embodiment, the electrode is independent of the headgear and allows for an easy and quick installation and removal of said electrode.

The present invention also relates to a process for the implementation of an electrode of the invention.

Said process comprises the following steps:
fastening the electrode to an electrode support in a predefined position;
applying pressure on the at least one fastener to deliver the electrolytic paste to the skin of a subject.

The electrode support is an EEG electrode support, an ECG electrode support, a FECG electrode support, an EMG electrode support, an EIT electrode support, a TENS electrode support, a bioimpedance electrode support, or any electrostimulation electrode support.

When a pressure is applied on the at least one fastener, such as for example a manual pressure applied with a finger, the foam material of the peripheral wall is deformed allowing the delivery of the electrolytic paste from the reservoir.

The delivery of the electrolytic paste from the at least one reservoir allows said paste to be in contact with the skin of the subject and therefore the recovery of the electrophysiological signal of interest. Once delivered, the electrolytic paste goes through the hair of the subject until it reaches the skin of said subject.

No preparation of the skin/scalp is required before setting the electrode on the body/head of a subject.

In case of an EEG electrode support, such as for example an EEG headgear, the electrolytic paste is delivered to the scalp of the subject.

In one embodiment, the process can be carried out by the subject himself, a third party or a medical staff.

In one embodiment, the predefined position refers to a position according to the International 10/20 System, 10/10 System, or any other positioning System.

### Definitions

In the present invention, the following terms have the following meanings:
“ECG” refers to electrocardiograph.
“EEG” refers to electroencephalograph.
“EIT” refers to electrical impedance tomography.
“Electrode support” refers to a rigid, flexible, stretchable or cloth part configured to handle at least one electrode in order to have them well placed on morphological points. The electrode support may be a garment.
“EMG” refers to electromyogram.
“FECG” refers to fetal electrocardiograph.
“Headgear” refers to an electrode support for the head.
“Paste” refers to a viscous liquid, a gel, a jellified liquid, or a cream.
“Snap fastener” refers to a pair of interlocking elements such as for example discs, made out of metal or plastic. A lip under one disc fits into a groove on the top of the other, holding them fast until a certain amount of force is applied. Snap fastener, snap button, press stud, popper, snap or stich are used interchangeably.
“TENS” refers to electrostimulation.

### DESCRIPTION OF THE DRAWINGS

The following detailed description will be better understood when read in conjunction with the drawings. For the purpose of illustrating, the electrode is shown in the preferred embodiments. It should be understood, however that the application is not limited to the precise arrangements, structures, features, embodiments, and aspect shown. The drawings are not drawn to scale and are not intended to limit the scope of the claims to the embodiments depicted. Accordingly, it should be understood that where features mentioned in the appended claims are followed by reference signs, such signs are included solely for the purpose of enhancing the intelligibility of the claims and are in no way limiting on the scope of the claims.

Features and advantages of the invention will become apparent from the following description of embodiments of an electrode according to the invention, this description being given merely by way of example and with reference to the appended drawings in which:

FIG. 1A is a schematic representation of an electrode according to a first embodiment of the invention before use.

FIG. 1B is a schematic representation of an electrode according to the first embodiment during use, i.e. when pressure is applied on the fastener.

FIG. 2A is a schematic representation of an electrode according to a second embodiment of the invention before use.

FIG. 2B is a schematic representation of an electrode according to the second embodiment during use, i.e. when pressure is applied on the fastener.

FIG. 3 is a schematic representation of an electrode according to a third embodiment of the invention before use.

FIG. 4A is a schematic representation of an electrode according to a fourth embodiment of the invention before use.

FIG. 4B is a schematic representation of an electrode according to the fourth embodiment during use, i.e. when pressure is applied on the fastener.

While various embodiments have been described and illustrated, the detailed description is not to be construed as being limited hereto. Various modifications can be made to the embodiments by those skilled in the art without departing from the true spirit and scope of the disclosure as defined by the claims.

### ILLUSTRATIVE EMBODIMENTS OF THE INVENTION

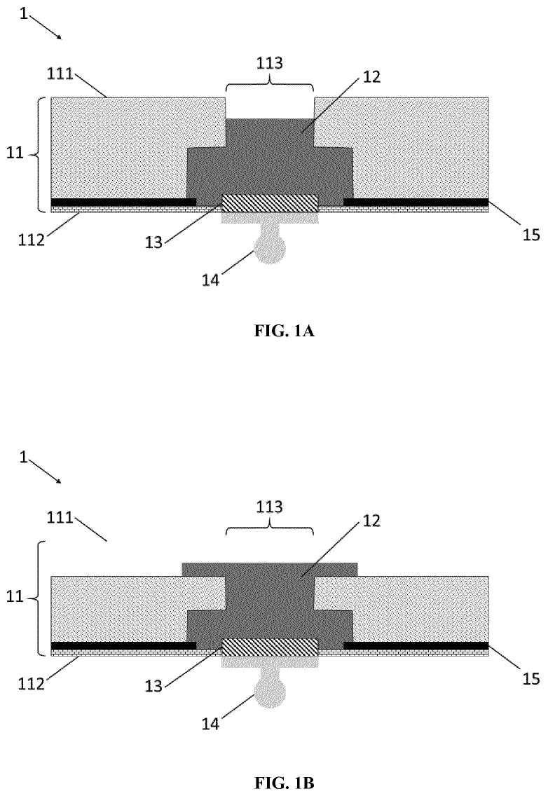

In the first embodiment shown in FIGS. 1A and 1B, the electrode 1 comprises:
a reservoir 11 comprising a peripheral wall 111 made of a foam material and a bottom polymeric washer 112, wherein said reservoir 11 is filled with an electrolytic paste 12;
a plate 13 comprising a conductor material, wherein said plate 13 is attached to the bottom polymeric washer 112 and in contact with the electrolytic paste 12; and
a fastener 14 attached to the bottom polymeric washer 112.

In this embodiment, the reservoir 11 comprises a hole 113 configured to release the electrolytic paste 12 under pressure applied on the at least one fastener 14.

This embodiment is particularly advantageous as the wear of electrode is comfortable due to the foam material, and the electrode 1 is ready to use and does not require any additional manipulation before being put to use in an EEG electrode support such as for example an EEG headgear, an ECG electrode support, a FECG electrode support, an EMG electrode support, an EIT electrode support, a TENS electrode support, a bioimpedance electrode support, or any electrostimulation electrode support.

As shown in FIG. 1A, before use, the electrolytic paste 12 resides in the reservoir 11. Said electrolytic paste 12 has a sufficiently high viscosity not to flow out of the reservoir 11 without an external mechanical action.

As shown in FIG. 1B, during use on the head of a subject, a pressure is applied on the fastener 14, resulting in a deformation of the peripheral wall 111, thus allowing to deliver the electrolytic paste 12 directly on the skin of the subject. The delivery of the electrolytic paste 12 from the at least one reservoir 11 allows said paste to be in contact with the skin of the subject and therefore the recovery of the signal. The applied pressure is manual.

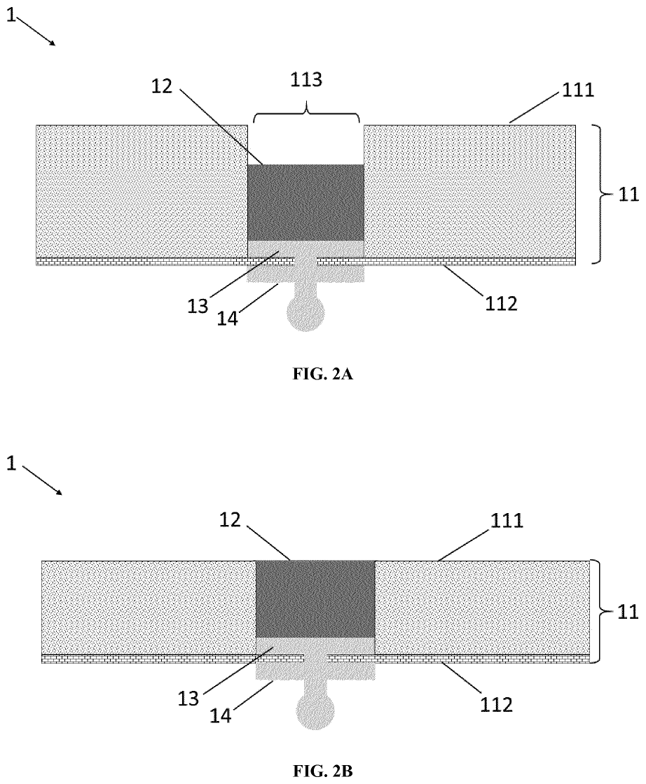

In the second embodiment shown in FIGS. 2A and 2B, elements similar to those of the first embodiment bear identical references. The electrode 1 of the second embodiment comprises:
a reservoir 11 comprising a peripheral wall 111 made of a foam material and a bottom polymeric washer 112, wherein said reservoir 11 is filled with an electrolytic paste 12;
a plate 13 comprising a conductor material, wherein said plate 13 is attached to the bottom polymeric washer 112 and in contact with the electrolytic paste 12; and
a fastener 14 attached to the bottom polymeric washer 112.

In this embodiment, the bottom polymeric washer 112 and the fastener 14 are combined.

In this embodiment, the reservoir 11 comprises a hole 113 configured to release the electrolytic paste 12 under pressure applied on the at least one fastener 14.

This embodiment is particularly advantageous as the wear of electrode is comfortable due to the foam material, and the electrode 1 is ready to use and does not require any additional manipulation before being put to use in an EEG electrode support such as for example an EEG headgear, an ECG electrode support, a FECG electrode support, an EMG electrode support, an EIT electrode support, a TENS electrode support, a bioimpedance electrode support, or any electrostimulation electrode support. Furthermore, as the bottom polymeric washer 112 and the fastener 14 are combined, there are less risk of separation of the electrode components.

As shown in FIG. 2A, before use, the electrolytic paste 12 resides in the reservoir 11. Said electrolytic paste 12 has a sufficiently high viscosity not to flow out of the reservoir without an external mechanical action.

As shown in FIG. 2B, during use on the head of a subject, a pressure is applied on the fastener 14, resulting in a deformation of the peripheral wall 111, thus allowing to deliver the electrolytic paste 12 directly on the skin of the subject. The delivery of the electrolytic paste 12 from the at least one reservoir 11 allows said paste to be in contact with the skin of the subject and therefore the recovery of the electrophysiological signal. The applied pressure is manual.

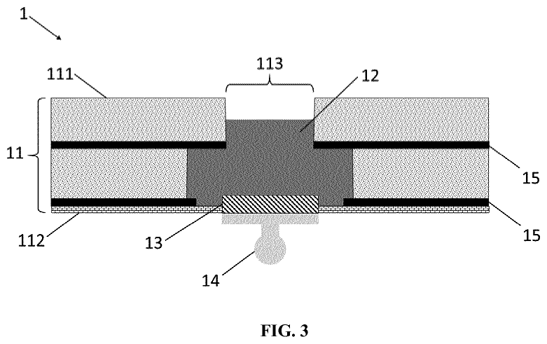

In the third embodiment shown in FIG. 3, elements similar to those of the first embodiment bear identical references. The electrode 1 of the third embodiment comprises:
a reservoir 11 comprising a peripheral wall 111 made of a foam material and a bottom polymeric washer 112, wherein said reservoir 11 is filled with an electrolytic paste 12;
a plate 13 comprising a conductor material, wherein said plate 13 is attached to the bottom polymeric washer 112 and in contact with the electrolytic paste 12; and
a fastener 14 attached to the bottom polymeric washer 112.

In this embodiment, the peripheral wall 111 comprises two parts assembled with a double-sided adhesive sheet 15. The advantage of this embodiment is the use of two layers of foam material that are 2D cut (laser cut, water cut, cutter cut, or punched, etc.) instead of a complex molding.

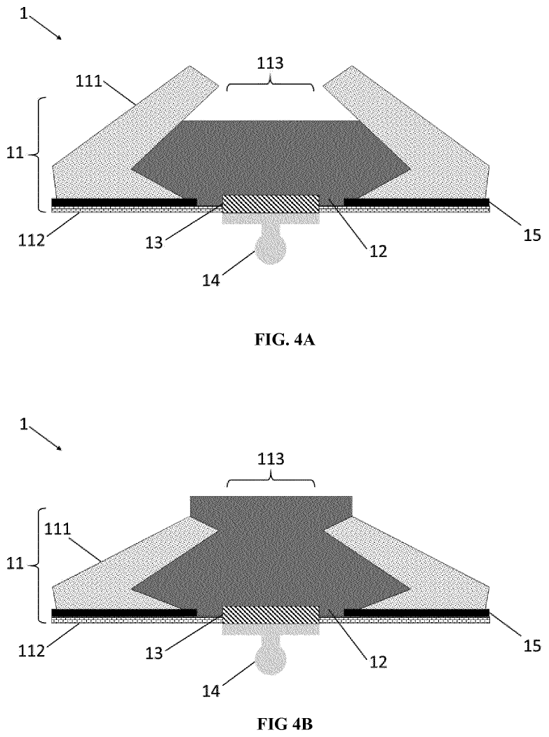

In the fourth embodiment shown in FIGS. 4A and 4B, elements similar to those of the first embodiment bear identical references. The electrode 1 of the fourth embodiment comprises:
a reservoir 11 comprising a peripheral wall 111 made of a foam material and a bottom polymeric washer 112, wherein said reservoir 11 is filled with an electrolytic paste 12;
a plate 13 comprising a conductor material, wherein said plate 13 is attached to the bottom polymeric washer 112 and in contact with the electrolytic paste 12; and
a fastener 14 attached to the bottom polymeric washer 112.

In this embodiment, the peripheral wall 111 is a cone. The operation of the electrode 1 is similar to that described in the first embodiment.

### REFERENCES

1—Electrode;
11—Reservoir;
111—Peripheral wall;
112—Bottom polymeric washer;
113—Hole;
12—Electrolytic paste;
13—Plate;
14—Fastener;
15—Double-sided adhesive sheet.

1. An electrode comprising:
at least one reservoir comprising a peripheral wall made of a foam material, wherein said reservoir is filled with an electrolytic paste, wherein the electrolytic paste exhibits a viscosity ranging from 10 Pa·s to 30 kPa s;
at least one plate comprising a conductor material, wherein said plate is in contact with the electrolytic paste and configured to conduct electrical signals to a recording device;
at least one fastener; and
wherein the at least one reservoir comprises a hole configured to release the electrolytic paste under pressure applied on the electrode.

2. The electrode according to claim 1, further comprising a bottom polymeric washer.

3. The electrode according to claim 1, further comprising at least one double-sided adhesive sheet between the peripheral wall and the bottom polymeric washer.

4. The electrode according to claim 1, wherein the at least one plate comprises a conductor material selected in the group of: Ag/AgCl, electrically conductive silicone, electrically conductive polymer, a plastic loaded with a conductive material such as for example a plastic coated with Ag/AgCl, or mixture thereof.

5. The electrode according to claim 1, wherein the at least one reservoir has a volume ranging from 0.1 ml to 100 ml.

6. The electrode according to claim 1, wherein the electrolytic paste exhibits an electrical conductivity ranging from 0.01 to 100 S/m.

7. The electrode according to claim 1, wherein the electrolytic paste exhibits a viscosity ranging from 10 kPa·s to 30 kPa·s.

8. The electrode according to claim 1, wherein the electrolytic paste exhibits a viscosity ranging from 10 Pa·s to 10 kPa·s.

9. The electrode according to claim 1, wherein the electrolytic paste is compatible with skin contact.

10. The electrode according to claim 1, wherein the at least one fastener is a snap fastener.

11. The electrode according to claim 1, wherein the foam material of the peripheral wall comprises a material selected in the group of: polyurethane, silicone, polyethylene, or a mixture thereof.

12. The electrode according to claim 1, wherein the foam material has at least one sticky face configured to adhere to the skin.

13. The electrode according to claim 1, wherein the peripheral wall comprises at least two parts assembled with a double-sided adhesive sheet.

14. The electrode according to claim 1, wherein said electrode is disposable.

15. The electrode according to claim 1, wherein said electrode is ready to use in an EEG electrode support such as for example an EEG headgear, an ECG electrode support, a FECG electrode support, an EMG electrode support, an EIT electrode support, a TENS electrode support, a bioimpedance electrode support, or any electrostimulation electrode support.

16. Use of the electrode according to claim 1 in an EEG electrode support, an ECG electrode support, an EMG electrode support, a FECG electrode support, an EIT electrode support, bioimpedance electrode support or any electrostimulation electrode support.

17. Process for the implementation of an electrode according to claim 1 comprising the following steps:
fastening the electrode to an electrode support in a predefined position; and
applying pressure on the at least one fastener to deliver the electrolytic paste from the at least one reservoir to the skin of a subject.
